(19) United States
(12) Patent Application Publication
(10) Pub. No.: US 2021/0228671 A1
(43) Pub. Date: Jul. 29, 2021

(54) EXTRACT OF VEGETATION WATERS FOR USE IN THE TREATMENT AND/OR IN THE PREVENTION OF PROSTATE CANCER

(71) Applicant: FATTORIA LA VIALLA DI GIANNI, ANTONIO E BANDINO LO FRANCO - SOCIETA' AGRICOLA SEMPLICE, Arezzo (IT)

(72) Inventors: Gianni LO FRANCO, Arezzo (IT); Antonio LO FRANCO, Arezzo (IT); Bandino LO FRANCO, Arezzo (IT); Adriana ALBINI, Sesto San Giovanni (Milano) (IT); Denisa BACI, Cavenago Di Brianza (Monza-Brianza) (IT); Antonino BRUNO, Milano (IT); Douglas NOONAN, Sesto San Giovanni (Milano) (IT); Matilde Elena TRAMACERE, Milano (IT)

(73) Assignee: FATTORIA LA VIALLA DI GIANNI, ANTONIO E BANDINO LO FRANCO - SOCIETA' AGRICOLA SEMPLICE, Arezzo (IT)

(21) Appl. No.: 17/053,568

(22) PCT Filed: Mar. 27, 2019
(86) PCT No.: PCT/IB2019/052476

§ 371 Date: Nov. 6, 2020

(30) Foreign Application Priority Data
May 8, 2018 (IT) 102018000005151

(51) Int. Cl. A61K 36/63; A61K 33/14; A61K 9/00; A61K 33/04; A61K 33/06; A61P 35/00; A61K 45/06; A61K 31/05; A61K 31/7048

(52) U.S. Cl. CPC A61K 36/63; A61K 33/14; A61K 9/0053; A61K 33/04; A61K 33/06; A61K 2236/51; A61P 35/00; A61K 45/06; A61K 31/05; A61K 31/7048; A61K 2236/53; A61K 9/0095

(57) ABSTRACT

The present invention relates to a natural phytocomplex or concentrate rich in polyphenolic compounds such as hydroxytyrosol and 3,4-DHPA-EDA derived from the waters from the pressing of olives for oil and/or pomace oil residues of the olive milling process, for use in the treatment and/or in the prevention of prostate carcinoma.

The present invention relates to a natural phytocomplex rich in polyphenolic compounds, in particular rich in hydroxytyrosol and oleuropein aglycone (3,4-DHPA-EDA), derived from the waters from the pressing of olives for oil (commonly known as vegetation waters) and/or pomace oil residues of the olive milling process, for use in the treatment and/or in the prevention of prostate cancer.

### PRIOR ART

One characteristic of olive oil that has aroused particular interest is the high content of polyphenols contained in it. These compounds are natural antioxidants of vegetable origin capable of inhibiting the formation of free radicals.

The beneficial properties of olive oil have resulted in a considerable increase, above all in Italy, in the cultivation of olive trees and the production of oil, with a consequent increase in the production of by-products of olive oil, mainly vegetation waters and pomace, which are characterized by a high pollutant load and thus generate a considerable environmental impact.

The disposal of this material is strictly regulated on both a national and regional level and the implementation of the legislation (law 574 of November 1996) imposes burdensome costs on producers, who are unable to derive any advantage from these waste products, which are, however, rich in molecules with a high medical/pharmaceutical potential.

Hydroxytyrosol constitutes the polyphenol that is present in the largest amount in vegetation waters and represents the most studied compound. It is present in vegetation waters and pomace and is also generated by the hydrolysis of oleuropein, a substance present above all in olive leaves.

Recent studies have demonstrated that hydroxytyrosol on its own has a cytoprotective effect vis-à-vis PC12 cells (a cell line of pheochromocytoma), is anti-apoptotic when administered to U937 cells (a human myelomonocytic line) and C2C12 cells (a line of mouse myoblasts), inhibits in vivo breast tumor proliferation in the case of induced neoplasms, is a chemopreventive agent in studies on HL60 and HL60R tumor lines (a line of human promyelocytic leukemia and the multi-drug resistant derivative thereof) and prevents premenstrual syndrome and osteoporosis. Furthermore, it has been demonstrated that the in vivo administration of hydroxytyrosol (also at high concentrations, up to 250-500 mg/kg/day) does not exert any toxic effect.

Other studies have demonstrated that oleuropein, when administered on its own, performs an antimicrobial activity, has an antitumoral potential in colorectal cancer cell lines, in metastatic breast cancer and in ER-negative breast cancer cell lines and has the ability to render the architecture of the cell cytoskeleton unstable.

Although many studies have been conducted on vegetation waters, there is still a greatly felt need to identify new properties that may lend value to these waste products, which would otherwise only represent a burdensome cost for the producer and an environmental contaminant.

Particularly felt is the need to identify new nutritional and/or medical/pharmacological properties that may raise the value of this waste product.

In this regard, the Applicant ha surprisingly found that vegetation waters are capable of performing a therapeutic and/or preventive effect on other forms of cancer in addition to the ones mentioned above, in particular on prostate cancer which, as is well known, represents the leading cause of cancer in the male population worldwide.

In particular, the Applicant has observed that, by concentrating, via reverse osmosis, the permeate of vegetation waters subjected to microfiltration, one obtains a phytocomplex rich in polyphenolic compounds for the treatment and/or prevention of prostate cancer, with a greater effectiveness than that of pure hydroxytyrosol, i.e. isolated from vegetation waters and/or from pomace by means of other purification techniques.

This therapeutic and/or chemopreventive effect is particularly advantageous for human health. In fact, the concentrate of vegetation waters, alone or in association with further substances with a known antitumor action against the various sub-forms of prostate cancer, can be used in therapy to treat such neoplasms.

### BRIEF DESCRIPTION OF THE FIGURES

Further advantages of the present invention will be apparent from the following detailed description and the appended figures, in particular:

FIG. 1 shows the results of the (3-(4,5-Dimethylthiazol-2-yl)-2,5-Diphenyltetrazolium Bromide (MTT) assay performed to evaluate the cell proliferation of two human prostate carcinoma cell lines (PC-3 and DU-145) treated with the polyphenolic concentrate of the present invention (sample A009) or with purified hydroxytyrosol (HyT), or with ethanol (EtOH, vehicle-control), at various dilutions. The assay was conducted 24, 48, 72 and 96 hours after treatment with each of the three compounds. NT=untreated cells; OD=optical density;

FIG. 2 shows the results of the in vitro fibronectin cell adhesion assay on human prostate carcinoma cell lines (PC-3 and DU-145) treated with the polyphenolic concentrate of the present invention (sample A009) or with purified hydroxytyrosol (HyT), or with ethanol (EtOH), all at a dilution of 1:500 or 1:250. NT=untreated cells;

FIG. 3 shows the results of the in vitro collagen cell migration assay (Boyden chamber) on human prostate carcinoma cell lines (PC-3 and DU-145) treated with the polyphenolic concentrate of the present invention (sample A009) or with purified hydroxytyrosol (HyT), or with ethanol (EtOH), all at a dilution of 1:500 or 1:250. NT=untreated cells; K−=(cells in serum−and growth factor-free medium);

FIG. 4 shows the results of the in vitro matrigel cell invasion assay (Boyden chamber) on cells of the human prostate carcinoma cell lines (PC-3 and DU-145) treated with the polyphenolic concentrate of the present invention (sample A009) or with purified hydroxytyrosol (HyT), or with ethanol (EtOH), all at a dilution of 1:500 or 1:250. NT=untreated cells; K−=(cells in serum−and growth factor-free medium);

FIG. 5 shows the results of the 7-aminoactinomycin D (7-AAD) assay performed to assess the percentage of apoptotic cells in the human cancer cell lines (PC-3 and DU-145) treated with the polyphenolic concentrate of the present invention (sample A009) or with purified hydroxytyrosol (HyT), or with ethanol (EtOH), at various dilutions. The assay was conducted 24 and 48 hours after treatment with each of the three compounds. NT=untreated cells;

FIG. 6 shows a cytofluorometric analysis of cytokine release in the human cancer cell lines (PC-3 and DU-145) treated with the polyphenolic concentrate of the present invention (sample A009) or with purified hydroxytyrosol (HyT), or with ethanol (EtOH), at various dilutions. NT=untreated cells;

FIG. 7 shows an analysis by secretome arrays of cytokine release in the human cancer cell lines (PC-3, DU-145 and LNCap) treated with the polyphenolic concentrate of the present invention (sample A009) at various dilutions. NT=untreated cells;

FIG. 8 shows the results of a flow cytofluorometric analysis of the cell cycle of the PC-3 cell line treated with the polyphenolic concentrate of the present invention (sample A009) or with purified hydroxytyrosol (HyT) at various dilutions or with vincristine 10 μM (Vin). The assay was conducted 24 and 48 hours after treatment with each of the three compounds. NT=untreated cells;

FIG. 9 shows the results of a flow cytofluorometric analysis of the cell cycle of the DU-145 cell line treated with the polyphenolic concentrate of the present invention (sample A009) or with purified hydroxytyrosol (HyT) at various dilutions or with vincristine 10 μM (Vin). The assay was conducted 24 and 48 hours after treatment with each of the three compounds. NT=untreated cells;

FIG. 10 shows the results of a flow cytofluorometric analysis of the cell cycle of the LNCap cell line treated with the polyphenolic concentrate of the present invention (sample A009) or with purified hydroxytyrosol (HyT) at various dilutions or with vincristine 10 μM (Vin). The assay was conducted 24 and 48 hours after treatment with each of the three compounds. NT=untreated cells.

### DETAILED DESCRIPTION OF THE INVENTION

The present invention relates to a phytocomplex or concentrate of vegetation waters and/or pomace comprising polyphenolic compounds, preferably hydroxytyrosol and/or 3,4-DHPA-EDA, for use in the treatment and/or in the prevention of prostate cancer. Said concentrate of vegetation waters and/or pomace has in fact demonstrated to be particularly effective in combating this type of cancer, by acting on various levels in combating tumor progression.

Hereinafter reference will be made to this phytocomplex or concentrate simply with the term “concentrate” or “polyphenolic concentrate”.

A second aspect of the present invention relates to a composition comprising the concentrate and further excipients/ingredients that are pharmacologically accepted for use in the treatment and/or in the prevention of cancer, in particular of prostate cancer.

In the context of the present invention, the expression “prostate cancer” means a benign or malignant neoplasm, which affects the gland. In particular, hereinafter reference will be made to prostate cancer.

The prostate is an organ belonging to the male genital apparatus, which intervenes in the production of seminal fluid. In an adult male, the prostate measures about three centimeters and weighs about twenty grams. The prostate contains many small glands that produce about twenty percent of the fluid part of semen. The functioning of the prostate is regulated by androgens, in particular testosterone, produced in the testicles, dehydroepiandrosterone, produced by the adrenal glands, and dihydrotestosterone, produced by the prostate itself.

In prostate carcinomas, the cells of the small glands making up the prostate change into cancerous cells. The specific causes of prostate cancer have not yet been fully identified. The most frequent form of prostatic neoplasia is acinar adenocarcinoma (which develops from the acinar structures of the prostate); other forms of prostatic neoplasia are, for example, ductal adenocarcinoma (which originates from the cells of the prostatic ducts), adenosquamous or squamous carcinoma, mucinous carcinoma, and small-cell carcinoma.

In a preferred embodiment of the invention, the concentrate and/or the composition according to the invention can be used for the treatment and/or prevention of malignant carcinomas of the prostate, in particular acinar adenocarcinoma. In fact, it has been surprisingly observed that the concentrate described herein possesses properties capable of combating tumor development and progression at various levels, and the use thereof is thus advantageous and effective, preferably in the treatment and/or in the prevention of prostatic neoplasms of a malignant type.

In fact, in vitro studies performed on cells of prostate cancer cell lines (specifically on cells of the lines PC-3 and DU-145) demonstrated that the concentrate according to the invention is capable, above all, of decreasing cell proliferation, with a greater effectiveness as compared to pure hydroxytyrosol.

Furthermore, the concentrate of the invention has demonstrated to be effective also in inhibiting in vitro the adhesion of cancer cells to a fibronectin matrix. This result may be related to the fact that the prostate cancer cells treated with the concentrate of the invention have a greater difficulty in originating metastasis due to the reduction in their ability to adhere and migrate on a fibronectin layer (component of the extracellular matrix) and invade (pass through) a basement membrane matrix (matrigel). Moreover, the in vitro studies (with a Boyden chamber) showed that the concentrate of the invention interferes with the migration and invasion capacity of prostate cancer cells. In particular, the treated cells are less able to migrate, suggesting an action of the compound on the cell migration pathway, by inhibiting the activation thereof or rendering it less efficient, thus reducing the ability of the tumor to propagate through the surrounding tissues.

Furthermore, the in vitro studies showed that the polyphenolic concentrate is capable of reducing the release of cytokines which favor tumor angiogenesis and inflammation, such as VEGF, CXCL8 (IL-8) and CXCL12 (SDF-1).

The in vitro studies on the prostate carcinoma cell lines further showed that the polyphenolic concentrate is capable of interfering with the cell cycle of prostate carcinoma cell lines.

The vegetation waters are preferably derived from a three-phase (oil, vegetation waters and pomace), and/or a two-phase (oil and pomace+vegetation waters) olive milling process. The vegetation waters generated by the mill can preferably be treated with an acidic pH solution which, preferably, ranges from 3 to 5; more preferably it is about 4 or 5. The pH is optimized, preferably by adding a strong acid, and/or pectolytic enzymes, i.e. enzymes that hydrolyze the cellulosic matrix of olive skins.

According to a preferred embodiment of the invention, the pomace is pitted, diluted and/or pre-filtered. The pomace preferably has a maximum particle size ranging from 0.5 to 1 millimeter (mm), more preferably about 0.7 mm. An example of a particle size is the one obtained by sieving with a vibrating screen. The pitted olive pomace may optionally be solubilized or dispersed in an aqueous matrix with a pH preferably comprised from 3 to 5, more preferably from 3.5 to 4.0.

The solubilization step has the purpose of solubilizing the polyphenols that would otherwise remain trapped in the solid matrix of the olive skins.

In a preferred embodiment of the invention, the concentrate further comprises: at least one further phenolic compound preferably selected from: tyrosol, chlorogenic acid, β-hydroxyverbascoide, rutin, verbascoide, and luteolin; and/or at least one metal preferably selected from: sodium, calcium, magnesium and potassium; and/or at least one anion preferably selected from: chlorides, sulphates, phosphates and nitrates; and/or at least one carbohydrate selected from: glucose, fructose, mannitol and sucrose.

In a further embodiment of the invention, the concentrate comprises nitrogenous substances (proteins, amino acids), preferably in an amount comprised from 15 to 60 mg/kg, more preferably from 20 to 40 mg/kg (mg of nitrogen per liter of active solution).

In any case, the phenolic compounds present in the concentrate in the largest amount are hydroxytyrosol and 3,4-DHPA-EDA.

The amount of hydroxytyrosol preferably ranges from 1 to 10 grams per liter of vegetation waters (g/L), more preferably from 1.5 to 5 g/L, even more preferably from 2 to 3 g/L.

The amount of 3,4-DHPA-EDA is preferably comprised from 0.5 to 8 g/L, more preferably from 1 to 6 g/L, even more preferably from 1.5 to 2.5 g/L.

The amount of tyrosol is preferably comprised from 0.1 to 0.4 g/L, more preferably from 0.15 g/L and 0.25 g/L.

The amount of chlorogenic acid is preferably comprised from 0.06 to 0.24 g/L, more preferably from 0.8 to 0.16 g/L.

The amount of tb-hydroxyverbascoide is preferably comprised from 0.3 to 1.5, more preferably from 0.5 to 1 g/L.

The amount of rutin is preferably comprised from 0.05 to 0.2 g/L, more preferably from 0.08 to 0.15 g/L.

The amount of verbascoside is preferably comprised from 0.4 to 1.7 g/L, more preferably from 0.6 to 1 g/L.

The amount of luteolin is preferably comprised from 0.1 to 0.5 g/L, more preferably from 0.15 to 0.28 g/L.

The amount of sodium is preferably comprised from 75 to 300 mg/L, more preferably from 120 to 180 mg/L.

The amount of calcium is preferably comprised from 5 to 10 g/L, more preferably from 2 to 5 g/L.

The amount of magnesium is preferably comprised from 220 to 900 mg/L, more preferably from 400 to 500 mg/L.

The amount of potassium is preferably comprised from 3 to 15 g/L, more preferably from 6 to 9 g/L.

The amount of chlorides is preferably comprised from 1.5 to 7 g/L, more preferably from 2.5 to 4.5 g/L.

The amount of sulphates is preferably comprised from 12 to 45 g/L, more preferably from 18 to 28 g/L.

The amount of phosphates is preferably comprised from 1.5 to 7 g/L, more preferably from 2.5 to 5 g/L.

The amount of nitrates is preferably comprised from 12 to 50 mg/L, more preferably from 18 to 30 mg/L.

The amount of glucose is preferably comprised from 15 to 60 g/L, more preferably from 25 to 35 g/L.

The amount of fructose is preferably comprised from 3.5 to 15 g/L, more preferably from 5 to 9 g/L.

The amount of mannitol is preferably comprised from 1 to 4 g/L, more preferably from 1.5 to 3 g/L.

The amount of sucrose is preferably comprised from 4 to 16 g/L, more preferably from 6 to 10 g/L.

In a preferred embodiment of the invention, the concentrate is obtained/obtainable by means of a process comprising the steps of: (i) microfiltering a sample of the vegetation waters and/or olive pomace so as to obtain a concentrate and a permeate of microfiltration; and (ii) concentrating by reverse osmosis the microfiltration permeate obtained from step (i).

The microfiltration is preferably performed after the solubilization step as described before.

The microfiltration has the purpose of separating a concentrate, i.e. the concentrated fraction of the content of the vegetation waters/pomace in suspension, for example micro fragments, fibers and corpuscular material such as cells and bacteria. It is carried out under the standard conditions for this type of matrix.

Following the microfiltration step, in addition to the concentrate, one obtains a permeate, i.e. a clear fraction, characterized by a color that varies according to the starting material and contains the dissolved components of the vegetation waters/pomace, e.g. proteins, sugars, salts, polyphenols, organic acids and various soluble organic molecules.

The microfiltration is preferably carried out with at least one, preferably two, ceramic membrane(s). The membrane is characterized by a preferably tubular shape. In a preferred embodiment the membrane is made of alumina oxide and/or zirconia.

The membrane preferably has the following characteristics: an outer diameter ranging from about 30 to about 40 mm, preferably of about 25 mm; and/or a length ranging from about 500 to about 1500 mm, preferably of about 1200 mm; and/or a series of channels with a diameter, preferably a hydraulic diameter, ranging from about 2.5 to about 5 mm, preferably of about 3.5 mm; and/or a filtering surface ranging from about 0.15 to about 0.7 m², preferably of about 0.35 m²; and/or a particle size or molecular weight cut-off ranging from about 0.1 micron to about 300 kDa.

The reverse osmosis step for concentrating the permeate obtained from the microfiltration of the vegetation water/pomace as described before is carried out under the standard conditions for this type of matrix, preferably by using a polymeric membrane, more preferably made of polyamide.

In particular, the membrane has a spiral-wound conformation and/or a molecular weight cut-off with high salt rejection, i.e. capable of rejecting sodium chloride molecules at a percentage of 99.9%. This means that the osmosis membrane holds back the molecules of biomedical interest and allows only water molecules to pass through.

The polymeric membrane preferably has a filtering surface ranging from about 5 to about 15 m², more preferably of about 7 m².

The reverse osmosis step enables the permeate obtained by microfiltration to be concentrated preferably by about 4 times; this means that from 100 L of microfiltration permeate 25 L of concentrate are obtained. In this case the volume concentration ratio (VCR) is 4, i.e. 100/25.

The VCR can change based on the starting matrix (vegetation waters) and above all based on its salt content, because the reverse osmosis process must offset the osmotic pressure of the matrix which is going to be concentrated.

The present invention further relates to a concentrate (or phytocomplex) of vegetation waters/olive pomace obtainable/obtained with the above-described process.

The concentrate preferably has the composition described before as regards the content of phenolic compounds, and/or metals, and/or carbohydrates, and/or anions and/or nitrogen.

According to a further aspect of the invention, the concentrate and/or composition as described above is/are used alone or in combination with other substances, compounds, drugs or compositions known to have anticancer effectiveness in the treatment of prostate cancer, in particular prostate carcinomas. Such other substances, compounds drugs or compositions can preferably be selected from vinca alkaloids and taxanes.

In one embodiment, the polyphenolic concentrate and/or the composition of the invention is/are advantageously used for the treatment and/or prevention of prostate cancer (prostate carcinomas). In one embodiment, the concentrate and/or the composition of the invention is/are used in the treatment and/or in the chemoprevention of acinar adenocarcinoma, which is the most common and widespread form of prostate cancer. In another embodiment, the concentrate and/or the composition of the invention is/are used in the treatment of small-cell carcinoma of the prostate, one of the most aggressive forms of prostate cancer.

In another aspect of the present invention, the concentrate of vegetation waters and/or pomace and/or the composition as described above is/are prepared in the form of an aqueous solution or emulsion or powder for injections for parenteral use, preferably for subcutaneous or intramuscular or intravenous use, more preferably for intravenous use. Said preparation for parenteral use can further comprise a vehicle, at least one additive selected from solubilizers, stabilizers, local anesthetics, preservatives, antibacterials, isotonifiers and mixtures thereof.

In another aspect of the present invention, the concentrate and/or composition as described above is/are in the form of a beverage. The beverage according to the invention can further comprise one or more optional excipients normally present in the formulation of various types of beverages. The beverage enriched with the concentrate and/or the composition as described above is definable as a functional type, i.e. to be used as a dietary supplement by virtue of the therapeutic effects found and described herein.

The beverage can preferably be water and/or fruit and/or milk-based. In a particularly preferred embodiment of the invention, the beverage is fruit-based, preferably grape-based. In particular, grape juice and/or must is preferred, preferably from organic grapes.

Alternatively, the concentrate and/or the composition can be prepared in a formulation for oral use of various types, e.g. pills, lozenges, tablets, or also powders or granules, preferably obtainable, for example, as a result of a drying and/or freeze-drying process.

In this case too, as for the beverage, the oral formulation is taken as a dietary supplement for the purpose of preventing prostate cancer, in particular prostate carcinomas.

Optionally, the beverage and/or the oral formulation is/are taken in association with one or more further substances, compounds, drugs or compositions known to have effectiveness against prostate cancer, as already described previously.

Optionally, the concentrate and/or the composition can also comprise further agents/molecules having a biologically relevant or adjuvant function with respect to the treatment of prostate cancer; for example, such other agents/molecules can have anti-inflammatory and/or antibiotic and/or antiangiogenic functions.

### EXAMPLES

Evaluation of the Effect of the Polyphenolic Concentrate on Cell Proliferation in Prostate Carcinoma Cell Lines.

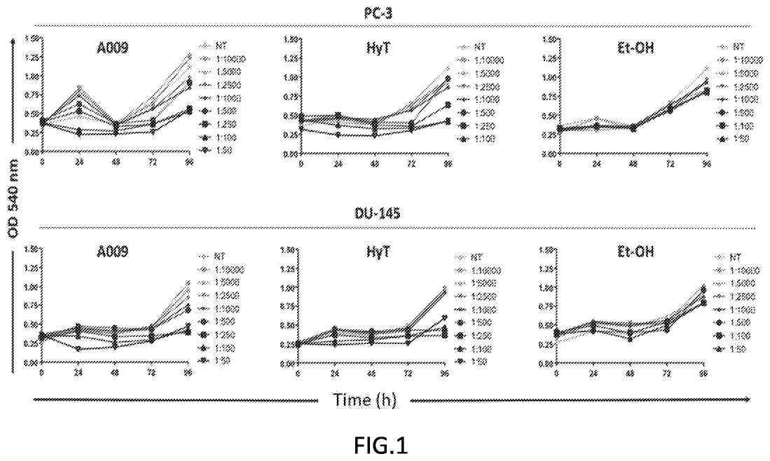

The effect of the polyphenolic concentrate of the invention (A009) on cell viability and proliferation was assessed by means of the MTT (tetrazolium salt, [3-(4,5-dimethylthiazol-2-yl)]-2,5-diphenyltetrazolium bromide) colorimetric viability assay on the cells of two prostate cancer cell lines (FIG. 1).

The MTT assay is based on the ability of the MTT compound to be metabolized by the mitochondrial enzyme succinate dehydrogenase. The reduction of the salt leads to the formation of crystals of a water-insoluble blue-colored product, formazan. Viable cells, unlike non-viable ones, reduce the salt and the amount of formazan produced is proportional to the number of cells presents. The crystals that form are solubilized and the absorbance (or optical density, OD) levels are determined by means of a spectrophotometer reading.

The cell models used are two human prostate cancer cell lines: PC-3 and DU-145. The cells of the PC-3 cell line are androgen-independent cells derived from prostate cancer bone metastasis and endowed with a high metastatic potential. The cells of the DU-145 cell line are derived from central nervous system metastasis; the metastatic potential of DU-145 cells is lower than that of PC-3 cells.

PC-3 and DU-145 cells were cultured and treated with the polyphenolic concentrate of the invention (A009), or with hydroxytyrosol (HyT), or with ethanol (EtOH); each substance was evaluated at the following dilutions: 1:10000-1:5000-1:2500-1:1000-1:500-1:100, 1:50; NT indicates the absence of any cell treatment.

The assays were conducted prior to treatment and 24, 48, 72 and 96 hours after treatment.

Cell viability (and consequently proliferation) is evaluated in terms of optical density (OD) measured by means of a spectrophotometer reading at 540 nm, that is, the optimal wavelength for evaluating cell proliferation based on the number of cells cultured and the pH of the cultural medium used (7.1-7.2).

With reference to the data shown in FIG. 1, it may be observed that the concentrate of the invention (A009) has an anti-proliferative effect on the two cancer cell lines. The effect of cell growth (understood as proliferation, or increase in the total number of cells in the culture) inhibition by A009 is dose-dependent and time-dependent.

Comparing the results obtained with A009 with those obtained with pure hydroxytyrosol (which represents the main component in the polyphenolic concentrate), it may be observed that these results are wholly comparable: it is thus significant that a concentrate of natural origin deriving from substances which substantially represent a waste product of the olive oil industry possesses noteworthy antioxidant properties, analogous to those of purified hydroxytyrosol. For this reason, the therapeutic and/or chemopreventive potential of the concentrate of the invention is extremely promising. The 1:250 and 1:500 dilutions of A009 were thus selected for subsequent functional studies.

Evaluation of the Effect of the Polyphenolic Concentrate on Cell Adhesion, Migration and Invasion In Vitro in Prostate Carcinoma Cell Lines.

An evaluation was made of the ability of the polyphenolic concentrate of the invention (A009) in combating some of the most common characteristics of cancer cells, i.e. the adhesion, migration and invasion capacity thereof, on which some fundamental processes for tumor progression depend, such as the generation of metastases from the primary tumor.

The cell models used are the two human prostate cancer cell lines PC-3 and DU-145 described in the previous example.

The above-mentioned characteristics were examined, respectively, by means of a fibronectin adhesion assay, collagen migration assay (using a Boyden chamber) and matrigel invasion assay (using a Boyden chamber). The cells were pretreated for 24 hours with the extract or pure hydroxytyrosol. For the adhesion assays, the cells were seeded on a fibronectin layer (2 g/mL) and incubated for 90 minutes. The adhering cells were stained with Dapi fluorescent dye and counted with a fluorescence microscope. For the migration and invasion assays, the cells were loaded into the upper compartment of the individual Boyden chambers. A polycarbonate filter (8 m pores), previously coated with fibronectin (2 g/mL) and matrigel (1 mg/mL), respectively, was interposed between the lower and upper chambers of the Boyden system. After 6 hours (migration) and 18 hours (invasion) of incubation, the filters were collected, stained with Dapi fluorescent dye and the cells were counted with a fluorescence microscope.

The PC-3 and DU-145 cells were cultured in vitro, then treated with the polyphenolic concentrate A009, or with hydroxytyrosol (HyT), or with ethanol (EtOH); each substance was evaluated at the 1:500 and 1:250 dilutions; NT indicates the absence of any treatment of the cells.

The effects were evaluated 24 hours after the treatment.

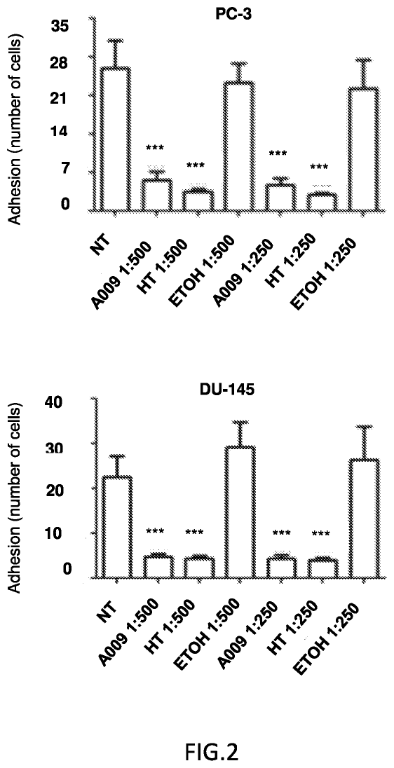

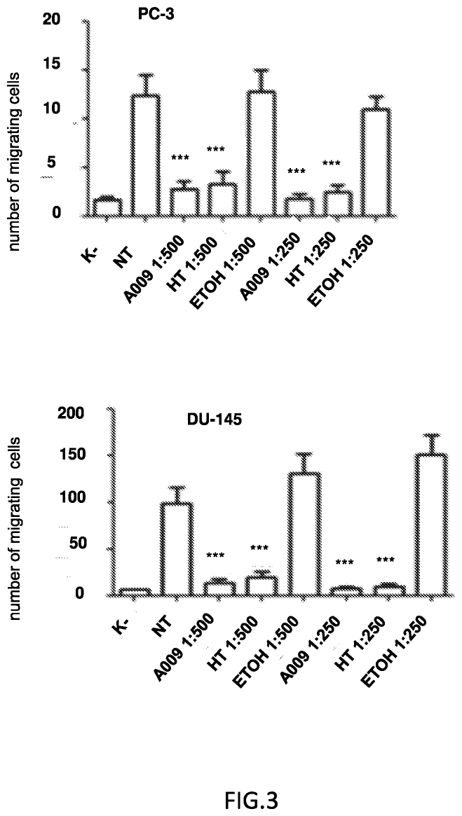

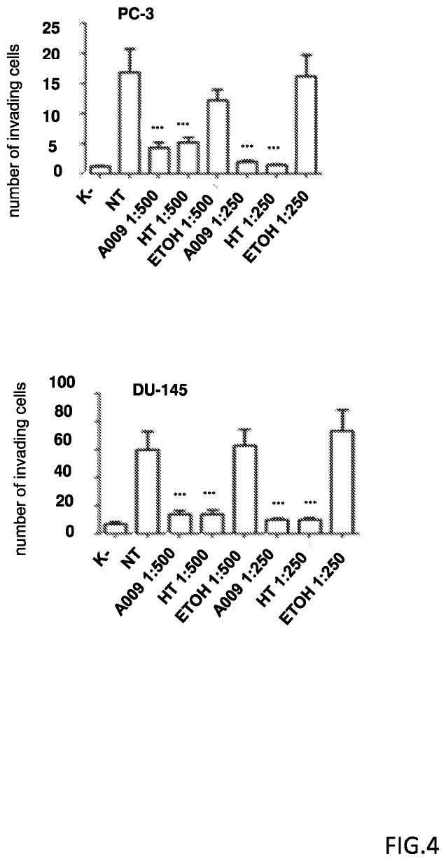

With reference to FIGS. 2, 3 and 4, it can be observed that the concentrate according to the invention inhibits the adhesion of the cells of both cancer lines on the fibronectin matrix (FIG. 2), and interferes with both cell migration (FIG. 3) and invasion in the Boyden chamber (FIG. 4). The extract A009 demonstrated greater activity than pure hydroxytyrosol.

Evaluation of the Pro-Apoptotic Activity of the Polyphenolic Concentrate In Vitro in Prostate Carcinoma Cell Lines.

The PC-3 and DU-145 cell lines were cultured in vitro, then treated with the polyphenolic concentrate A009, or with hydroxytyrosol (HyT), or with ethanol (EtOH); each substance was evaluated at the 1:500 and 1:250 dilutions; NT indicates the absence of any treatment of the cells. 7-AAD (7-Aminoactinomycin D) is a fluorescent cell viability dye which is excluded from live cells with intact membranes, but penetrates into dead or damaged cells and binds with double-stranded DNA with high affinity by intercalating between GC base pairs.

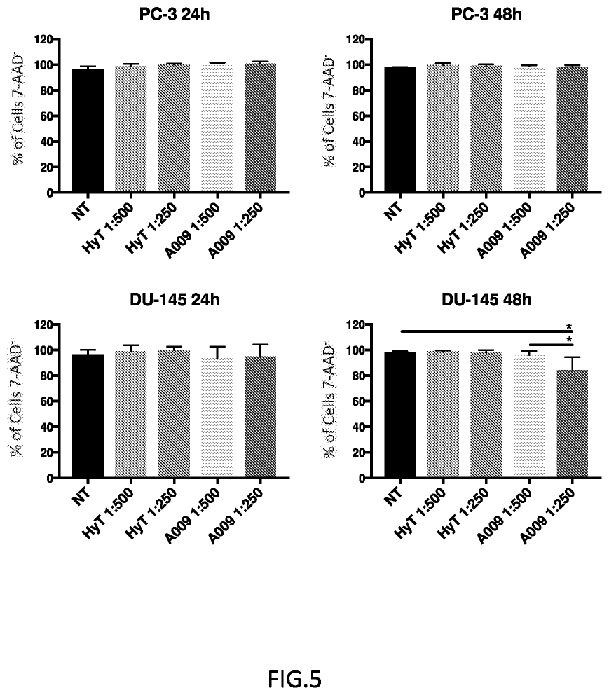

With reference to FIG. 5, it may be observed that the concentrate according to the invention does not show a significant pro-apoptotic activity, compared to untreated cells, on the PC-3 cell line at either the 1:500 dilution or the 1:250 dilution, 24 or 48 hours after the start of treatment.

However, the concentrate shows a significant variation in pro-apoptotic activity, compared to untreated cells, cells treated with hydroxytyrosol and cells treated with the concentrate at the 1:500 dilution, on the DU-145 cell line 48 hours after the start of treatment at the 1:250 dilution. This result demonstrates a greater sensitivity on the part of the DU-145 line, suggesting that the origin of the cancer plays a role in the response sensitivity to the substances administered.

Evaluation of the In Vitro Modulation, by the Polyphenolic Concentrate, of Cytokine Release in Prostate Cancer Cells.

The PC-3 and DU-145 cell lines were cultured in vitro, then treated with the polyphenolic concentrate A009 or with hydroxytyrosol (HyT), or with ethanol (EtOH); each substance was evaluated at the 1:500 and 1:250 dilutions; NT indicates the absence of any treatment of the cells.

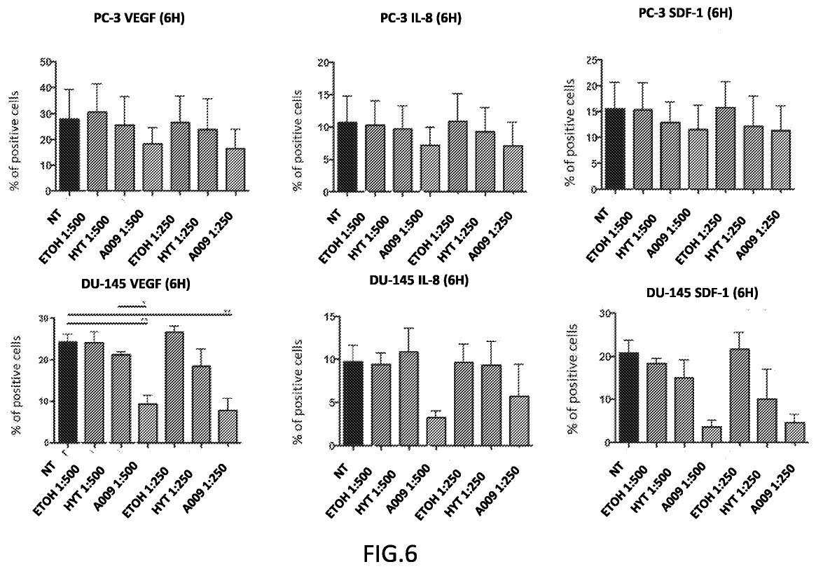

It was then evaluated, by flow cytofluorometry, whether the extract A009 is capable of modulating the release of pro-angiogenic and pro-inflammatory cytokines (VEGF, CXCL/8IL-8, CXCL12/SDF-1) by prostate carcinoma cells (FIG. 6). In detail, the concentrate shows a general trend towards decreasing the levels of VEGF, CXCL8/IL-8 and CXCL12/SDF in DU-145 cells after 6 hours of treatment, both at the 1:250 dilution and at the 1:500 dilution.

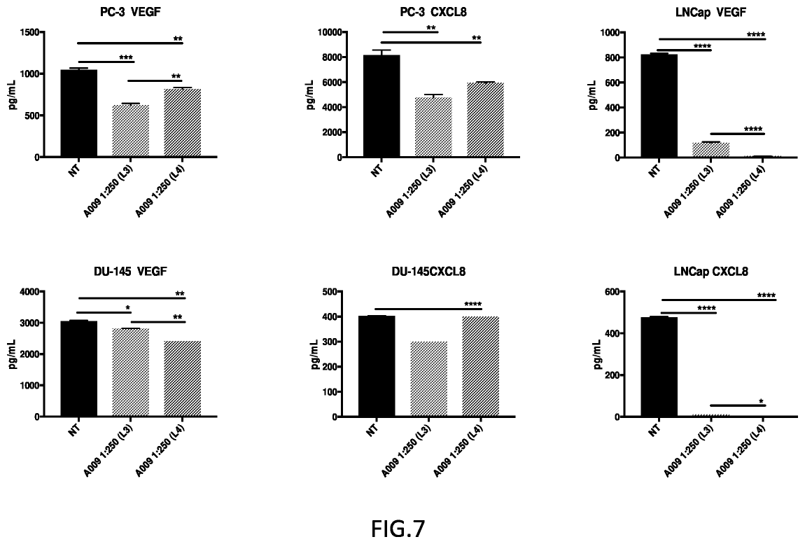

The data obtained by means of flow cytometry were validated and extended to a broader panel of cytokines using secretome arrays that rely on Bio-Plex technology, characterized by greater sensitivity. Supernatants obtained from the two prostate carcinoma cell lines (PC-3, DU-145), after 24 hours treatment with two different batches of the extract A009 (in serum−and growth factor-free medium) were subsequently analyzed by means of the BIO-PLEX platform, capable of measuring cytokine levels, with high sensitivity, by chemiluminescence. An evaluation was made, furthermore, of the ability of the extract in modulating the release of VEGF and CXCL8/IL-8 in a third cell line, LNCap (FIG. 7). LNCap cells are a human cell line commonly used in the field of oncology. LNCaP cells are androgen-sensitive human prostate adenocarcinoma cells derived from the left supraclavicular lymph node metastasis from a 50-year-old Caucasian male in 1977. They are adherent epithelial cells that grow in aggregates and as individual cells. The data obtained show that the extract A009 is capable of interfering with the release of pro-inflammatory and pro-angiogenic cytokines by three different prostate carcinoma lines.

Evaluation of the Ability of the Polyphenolic Concentrate to Halt the Cell Cycle in Prostate Cancer Cell Lines In Vitro.

The PC-3, DU-145 and LNCap cell lines were cultured in vitro, then treated with the polyphenolic concentrate A009, or with hydroxytyrosol (HyT). Each substance was evaluated at the 1:500 and 1:250 dilutions; NT indicates the absence of any treatment of the cells. The agent vincristine (10 μM), capable of inducing apoptosis, was used as a positive control.

The cell cycle was evaluated by flow cytofluorometry using propidium iodide (PI) as the DNA intercalating agent. In fact, through cytofluorometric analysis with a DNA intercalating agent it is possible to determine which phase of the cell cycle a cell is currently in. Under normal conditions, all healthy diploid cells are in the G0/G1 phase of the cell cycle and, in the same eukaryotic organism, should have the same amount of DNA (2n). The synthesis of DNA during the S phase of the cycle results in an increase in the cellular DNA content, which reaches a value of 4n and remains unchanged during the G2 phase and during mitosis (M), at the end of which the original cell is divided into two daughter cells, each with a content 2n of nucleic acid.

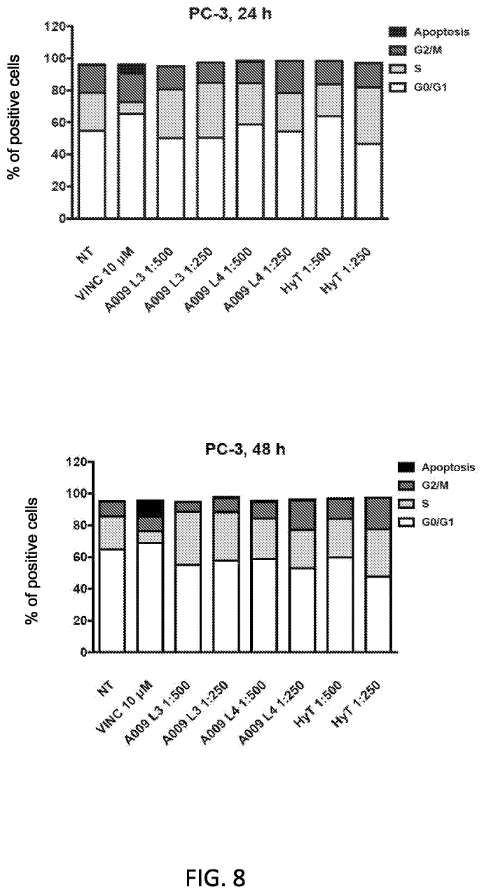

With reference to FIG. 8, the polyphenolic concentrate does not interfere with the cell cycle of the PC-3 cells in any phase of the cell cycle.

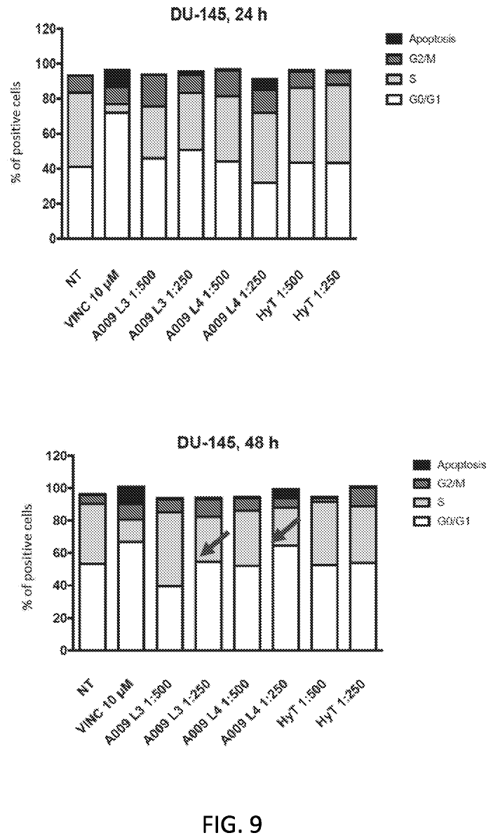

With reference to FIG. 9, the polyphenolic concentrate is capable of interfering with the S phase of the cell cycle of DU-145 cells after 48 hours of treatment at the 1:250 dilution.

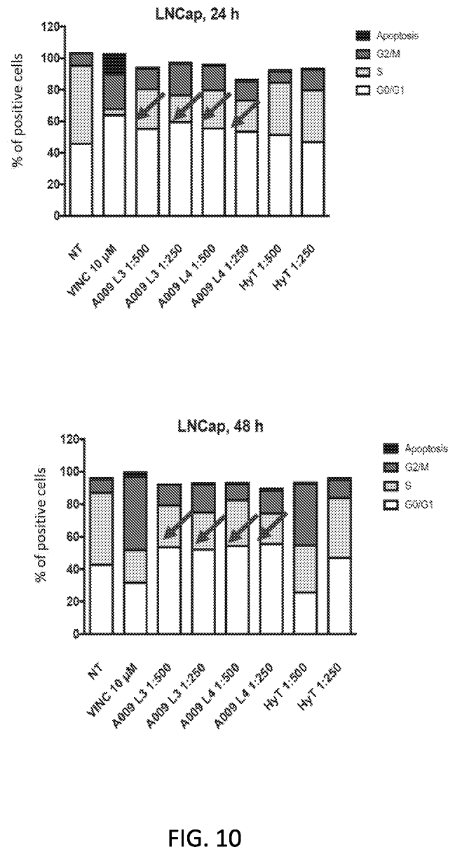

With reference to FIG. 10, the polyphenolic concentrate is capable of interfering with the S phase of the cell cycle of LNCap cells after 24 and 48 hours of treatment at the 1:250 and 1:500 dilutions. These results show a different sensitivity of the 3 cell lines, indicating that hormone sensitivity (LnCAP cells) is a factor capable of rendering prostate carcinoma cells more sensitive to the effect of the extract A009.

1. A method for treating and/or preventing prostate cancer, comprising administering to a subject a concentrate of vegetation waters and/or olive pomace comprising hydroxytyrosol and 3,4-DHPA-EDA.

2. The method of claim 1, wherein said concentrate further comprises:
at least one phenolic compound; and/or
at least one metal; and/or
at least one anion; and/or
at least one carbohydrate; and/or
nitrogen.

3. The method of claim 1, wherein said concentrate is obtained by means of a process comprising the steps of:
(i) microfiltering a sample of the vegetation waters and/or olive pomace so as to obtain a concentrate and a permeate of microfiltration; and
(ii) concentrating by reverse osmosis the microfiltration permeate obtained from step (i).

4. The method of claim 3, wherein the microfiltration step involves the use of at least one ceramic membrane.

5. The method of claim 3, wherein the reverse osmosis is performed by using a polymeric membrane.

6. The method of claim 1, wherein said prostate cancer is a prostate carcinoma wherein said prostate carcinoma is acinar adenocarcinoma.

7. The method of claim 1, wherein the concentrate is administered alone or in combination or in association with one or more other substances, compounds, drugs or compositions known to have anticancer effectiveness in the treatment of prostate cancer.

8. The method of claim 1, wherein said concentrate is formulated as an aqueous solution or emulsion or powder for injections for parenteral use.

9. The method of claim 1, wherein said concentrate is a formulation for oral use selected from the group consisting of pills, tablets, powders and granules.

10. The method of claim 1, wherein said concentrate is in the form of a beverage.

11. The method of claim 8, wherein said parenteral use is selected from the group consisting of subcutaneous use, intramuscular use, or intravenous use.

12. The method of claim 9, wherein said powders and granules are obtained by drying and/or freeze-drying.

13. The method of claim 10, wherein said beverage, is water-based and/or fruit-based and/or milk-based.

14. The method of claim 13, wherein said beverage is based on grape juice or must.

15. The method of claim 2, wherein
the at least one phenolic compound is selected from the group consisting of tyrosol, chlorogenic acid, β-hydroxyverbascoide, rutin, verbascoide, and luteolin; and/or
the at least one metal is selected from the group consisting of sodium, calcium, magnesium and potassium; and/or
the at least one anion is selected from the group consisting of chlorides, sulphates, phosphates and nitrates; and/or
the at least one carbohydrate is selected from the group consisting of glucose, fructose, mannitol and sucrose.

16. The method of claim 4, wherein the at least one ceramic membrane is characterized by a tubular shape.

17. The method of claim 4, wherein the at least one ceramic membrane is made of aluminum oxide and zirconia.

18. The method of claim 5, wherein the polymeric membrane is made of polyamide.

19. The method of claim 5, wherein the polymeric membrane is characterized by a spiral shape.
